SEQUENCE LISTING (24 sequences)

SEQ ID NO: 1 - protein, 16 aa, synthetic construct
GTRLKPLIICVQWPGL

SEQ ID NO: 2 - protein, 16 aa, synthetic construct
AISLHPKAKILEWPGA

SEQ ID NO: 3 - protein, 16 aa, synthetic construct
PLDIRDRVHVEKSAAS

SEQ ID NO: 4 - protein, 16 aa, synthetic construct
SVAVNPTPTLMDAPGG

SEQ ID NO: 5 - protein, 16 aa, synthetic construct
SVAVNDTPTLMDAAAG

SEQ ID NO: 6 - protein, 16 aa, synthetic construct
FHTLEPSLLAINTPGV

SEQ ID NO: 7 - protein, 16 aa, synthetic construct
FHTIEDSLLAINTAAV

SEQ ID NO: 8 - protein, 16 aa, synthetic construct
FHTVEPSLLAINTPGV

SEQ ID NO: 9 - protein, 16 aa, synthetic construct
GTRIKDLIICVQSAAL

SEQ ID NO: 10 - protein, 16 aa, synthetic construct
GTRVKPLIICVQAPGL

SEQ ID NO: 11 - protein, 16 aa, synthetic construct
LIELHPAARITEWPGA

SEQ ID NO: 12 - protein, 16 aa, synthetic construct
LLEIRDAVRVTKSAAS

SEQ ID NO: 13 - protein, 16 aa, synthetic construct
VISLHDKAAIHEWPGA

SEQ ID NO: 14 - protein, 16 aa, synthetic construct
VLDIRPRVAVHKSAAA

SEQ ID NO: 15 - protein, 21 aa, Mus musculus
METDTLLLWVLLLWVPGSTGD

SEQ ID NO: 16 - protein, 8 aa, synthetic construct
AAQPARRA

SEQ ID NO: 17 - protein, 9 aa, Homo sapiens
EQKLISEED

SEQ ID NO: 18 - protein, 10 aa, Homo sapiens
EQKLISEEDL

SEQ ID NO: 19 - protein, 54 aa, synthetic construct
METDTLLLWVLLLWVPGSTGDAAQPARRAEQKLISEEDGTRLKPLIICVQWPGL

SEQ ID NO: 20 - DNA, 135 bp, synthetic construct
  source 1..135: mol_type = other DNA
atggaaactgatactctactactatgggttctactactatgggttccaggtagtactggtgatgcagcacaacctgcaagaagagcaggtacccgtctaaaaccactaattatttgtgttcaatggccaggtcta

SEQ ID NO: 21 - protein, 33 aa, synthetic construct
AAQPARRAEQKLISEEDGTRLKPLIICVQWPGL

SEQ ID NO: 22 - protein, 16 aa, synthetic construct
VLIKIPWLRLPGCGTQ

SEQ ID NO: 23 - DNA, 20 bp, synthetic construct
  source 1..20: mol_type = other DNA
tatcagcgaggaggacggta

SEQ ID NO: 24 - DNA, 20 bp, synthetic construct
  source 1..20: mol_type = other DNA
cagatgatcaggggcttcag

(19) United States
(12) Patent Application Publication
(10) Pub. No.: US 2025/0066420 A1
(43) Pub. Date: Feb. 27, 2025

(54) PD-L1 INHIBITORY PEPTIDE FOR CANCER IMMUNOTHERAPY

(71) Applicant: Kansas State University Research Foundation, Manhattan, KS (US)

(72) Inventors: Jeffrey COMER, Manhattan, KS (US); Ettayapuram Ramaprasad Azhagiya SINGAM, Manhattan, KS (US); Masaaki TAMURA, Manhattan, KS (US); Susumu ISHIGURO, Manhattan, KS (US)

(21) Appl. No.: 18/696,155

(22) PCT Filed: Sep. 27, 2022
(86) PCT No.: PCT/US2022/077065

§ 371 Date: Mar. 27, 2024

Related U.S. Application Data
(60) Provisional application No. 63/248,939, filed on Sep. 27, 2021.

(51) Int. Cl. C07K 7/08; A61K 9/00; A61K 38/00; A61P 35/00; C12N 15/86

(52) U.S. Cl. CPC C07K 7/08; A61K 9/0019; A61P 35/00; C12N 15/86; A61K 38/00; C12N 2710/10043

(57) ABSTRACT

An immune checkpoint (PD-L1) inhibitor oligopeptide comprising the sequence GTRLKPLIICVQWPGL (SEQ ID NO:1), nucleic acids encoding the oligopeptide, and vectors and cells for delivery nucleic acids encoding the oligopeptide are disclosed. Methods of treating cancer and enhancing anti-tumor immunity are also disclosed.

### CROSS-REFERENCE TO RELATED APPLICATIONS

The present application claims the priority benefit of U.S. Provisional Patent Application Ser. No. 63/248,939, filed Sep. 27, 2021, entitled PD-L1 INHIBITORY PEPTIDE FOR CANCER IMMUNOTHERAPY, incorporated by reference in its entirety herein.

### FEDERALLY SPONSORED RESEARCH OR DEVELOPMENT

This invention was made with government support under 1 R15 CA219919-01 awarded by the National Institutes of Health, and DMR-1945589 awarded by the National Science Foundation. The government has certain rights in the invention.

### SEQUENCE LISTING

The following application contains a sequence listing submitted as an ST.26-compliant XML file entitled “Sequence_Listing_56294-PCT.xml” created on Sep. 23, 2022, as 21,941 bytes, the contents of which are incorporated by reference herein.

### BACKGROUND

### Field of the Invention

The present invention relates to immune checkpoint inhibitor peptides for treatment of cancer and enhancement of immune response to cancer.

### Description of Related Art

The cytotoxic T-lymphocyte-associated antigen 4 (CTLA-4) and programmed death 1 (PD-1, aka CD279) immune checkpoints are negative regulators of T-cell immune function. They prevent the immune system from attacking cells indiscriminately and play an important role in self-tolerance and deactivation of immune responses. The CTLA-4 and PD-1 immune checkpoint pathways downregulate T-cell activation to maintain peripheral tolerance.

However, immune checkpoint pathways are also one of the mechanisms by which cancer cells escape from surveillance of host anticancer immunity. Cytotoxic CD8 T lymphocytes (CTL), which play an important role in spontaneous inhibition of cancer development, attempt to remove cancer cells from their immune environment. Protein programmed cell death-1 ligand (PD-L1, as well as PD-L2) are natural ligands for PD-1 expressed by T cells, B cells, dendritic cells, and monocytes. PD-L1 is normally expressed by T cells, B cells, NK cells, dendritic cells, macrophages, MDSCs, and many other cell types such as epithelial and endothelial cells. PD-L1 are also expressed abundantly in tumor cells. PD-1 and PD-L1 binding is often a hallmark of CTL exhaustion and upregulation of surface PD-L1 on tumor cells has been shown to be associated with immune evasion and resultant aggressive tumor growth. The interaction of PD-L1 with PD-1 causes functional inactivation of CTL allowing cancer cells to survive. PD-L1 stimulates the immune checkpoint mechanism by interacting with PD-1 molecules expressed on T cells, attenuating cytotoxic T cell-dependent oncolysis. PD-1/PD-L1 expression downregulates the T cell immune response, allowing the cancer cells to evade the immune system, making this pathway an attractive target for immunotherapy.

Anticancer immunity plays an important role in spontaneous inhibition of cancer growth. The success of immunotherapy, which has been established in the last decade as a treatment against multiple immunogenic cancers, including non-small cell lung cancers and colorectal cancer, supports this notion. However, current immunotherapies targeting the PD-1/PD-L1 pathway rely on systemic inhibition of PD-L1 or PD-1, resulting in immune-related adverse events in 12-37% of patients treated, some of which are life-threatening.

Therefore, the development of safer, less toxic and more effective enhancers of anticancer immunity would be beneficial for both cancer patients as well as society at large. Currently, the most common strategy for cancer immunotherapy is systemic administration of humanized antibodies against immune checkpoint proteins, such as CTLA-4 and PD-1. Although these antibody-based therapeutics for cancer immunotherapy exhibit solid efficacy against immunogenic cancers, their off-target effects due to an enhancement of host immunity remain problematic. For example, long course treatment with anti-PD-1/PD-L1 antibodies has been shown to associate with immune-related adverse events (irAE).

In the United States, lung cancer is by far the leading cause of cancer death, making up almost 25% of all cancer deaths. Common therapies for lung cancer include immunotherapy, surgical resection, chemotherapy, and radiation therapy. Colorectal cancer (CRC) is the second leading cause of cancer death in both sexes combined and there were an estimated 104,270 new cases and 52,980 deaths in 2021. Because of improvements in early detection and treatment, the current five-year survival rate is 90% in patients diagnosed with early-stage CRC. However, survival rates of patients diagnosed with regional and distant metastases are 72% and 15%, respectively. Therefore, CRC still comprises a significant portion of cancer-dependent mortality and morbidity. Accordingly, novel cancer prevention and therapeutic strategies for both primary and metastatic CRC and lung cancer are urgently needed.

Given that current strategies for cancer treatment have limited success, immune checkpoint blockade therapy has emerged as a powerful new tool for cancer therapy. However, only CRCs associated with microsatellite instability (MSI) or DNA mismatch repair gene defects (dMMR, approximately 15% of all CRCs) are sensitive to this therapy and systemic side effects remain major concerns. This poor sensitivity to immune checkpoint blockade therapy is either due to poor tumor infiltration of functional T cells or T cell exhaustion. A combination treatment with an oncolytic virus and an immune checkpoint inhibitor is found to be very effective in increasing therapeutic efficacy and tumor immunogenicity since the oncolytic virus infection exposes neoantigens and causes additional T cell infiltration into the tumor tissue. However, an oncolytic virus is a pathogen and systemic administration of oncolytic virus induces a robust host immune response against virus and unwanted side effects.

Therefore, novel strategies to inhibit immune checkpoint between cancer cells and immune cells are urgently required.

### SUMMARY

The present invention is broadly concerned with immune checkpoint inhibitor peptides (oligopeptides) and methods related to treatment of any cancer in which inhibition of PD-L1 has been shown to improve treatment outcomes, including where inhibition of PD-L1 has been shown effective in stimulating antitumor responses.

In one or more embodiments, the methods relate to treatment of patients which are found to exhibit PD-L1 expression using standard assays. Embodiments described herein include immune checkpoint inhibitor oligopeptides designated as PD-L1ip3 and comprising (consisting essentially or even consisting of) the sequence:

GTRLKPLIICVQWPGL (SEQ ID NO:1)

Also disclosed herein are nucleic acids encoding the immune checkpoint inhibitor oligopeptide, as well as vectors comprising the nucleic acids and cells containing the same. In one or more embodiments, therapeutic compositions are disclosed which comprise a therapeutically effective amount of an immune checkpoint inhibitor oligopeptide, a nucleic acid, a viral vector, or a cell according any of the embodiments disclosed herein.

The present disclosure also concerns methods of treating cancer in a subject in need thereof by administering a therapeutically effective amount of an immune checkpoint inhibitor oligopeptide, a nucleic acid, a viral vector, or a cell according any of the embodiments disclosed herein. In one or more embodiments, local immune checkpoint inhibition coupled with some oncolysis may be an ideal immune checkpoint blockade therapy. Hence, a more local blockade of PD-L1, restricted to cancer tissue, would be desirable, with less potential for off-target effects. Thus, the present disclosure explores PD-L1-targeted immune checkpoint blockade along with mild oncolysis in cancer tissue as a local immune checkpoint blockade therapy, and utilizes a novel approach to PD-1/PD-L1 blockade via an inhibitory peptide which can be administered specifically at the tumor site, thus limiting the adverse systemic effects of antibody-mediated immune checkpoint inhibition. The short oligopeptide specifically interacts with the binding site of PD-L1 leading to upregulation of CTLs and enhanced anti-tumor immunity and stimulating cytotoxic T cell-induced cell death in cancer cells.

The immune checkpoint inhibitor peptides can be used for novel cancer immunotherapies. The immune checkpoint inhibitor peptides bind with the PD-L1 and blocks its association with PD-1 on CTLs. That is, the immune checkpoint inhibitor peptides bind the PD-L1 on target cancer cells interfering with their ability to bind to PD-1 such that an immune response can be triggered against cancer.

The present disclosure also describes kits comprising a plurality of immune checkpoint inhibitor oligopeptides, a nucleic acid, a viral vector, or a cell according to the various embodiments described herein in a unit dosage form in a container, and instructions for administering the therapeutic preparations to a subject in need thereof.

Also described herein are medicaments for use in treating cancer, inhibiting growth of cancer cells, or enhancing cancer treatment, comprising an immune checkpoint inhibitor oligopeptide, a nucleic acid, a viral vector, or a cell according to the various embodiments described herein.

The ability of PD-L1ip3 peptide to stimulate cancer cell antigen-primed CD8⁺ T cells to induce cell death in colon carcinoma cells is demonstrated via working examples. Moreover, the disclosure proposes a combination treatment of direct administration of PD-L1ip3 in peptide form along with viral vector gene therapy encoding the same peptide. These results clearly indicate a practical possibility of tumor cell-targeted gene therapy with a PD-L1 inhibitory peptide gene along with direct peptide administration. Moreover, the peptide shows no cytotoxicity while activating CD8⁺ T cells, and represents a significant advancement in cancer treatment options.

### BRIEF DESCRIPTION OF FIGURES

FIG. 1 shows a graph of PD-L1ip3 gene expression in Ad-PD-L1ip3 transduced CT26 cells. Expression of PD-L1ip3 gene was measured by RT-qPCR at Day 3, 5 and 7 after transduction in duplicate measurements.

FIG. 2A is an image depicting binding of peptide PD-L1ip3 to PD-L1 and the predicted lowest free energy binding conformation.

FIG. 2B is an image showing atomic interactions between PD-L1ip3 and PD-L1 stabilizing binding in this conformation. Carbon atoms of PD-L1ip3 (G1, L16, R3) are shown in green, while those of PD-L1 (M59, N63, E58, D73, E71, V68, R113, R125, and Y123) are shown in gray.

FIG. 2C shows a graph of the kinetics of binding between the designed peptide (PD-L1ip3) and PD-L1 during the association and dissociation phases of bio-layer interferometry experiments for different concentrations of PD-L1ip3. The lines correspond to PD-L1ip3 concentrations of 0, 1.71, 8.54, 17.1 and 171 μM, respectively, as labeled.

FIG. 3 shows graphs showing the viability of CT26 cells was not affected by treatment with PD-L1ip3 or PD-L1ip3SC (the scrambled sequence control) in peptide form (panel A) or adenovirus vector encoding PD-L1ip3 or PD-L1ip3SC gene (panel B) in the presence of mIFNγ. The mIFNγ (25 ng/ml) was added to the culture medium 24 h prior to the addition of the PD-L1ip3 or PD-L1ip3SC in peptide form (panel A, μg/ml) or Ad-PD-L1ip3 or Ad-PD-L1ip3SC transduction (panel B. MOI). PBS was added as a control for peptide or adenovirus vector treatment. Viability of CT26 cells was determined using MTT assay. Results are presented as mean±SD (n=3).

FIG. 4 shows graphs showing the treatment with PD-L1ip3 in peptide form significantly increased T cell-induced death of CT26 cells. (A) The most effective CT26 cell antigen-primed T cell (CT26-T cell) induced death in co-cultured CT26 cancer cells, as determined by varying the ratio of CT26 cells to these antigen-primed T cells (1:4-1:24). The two types of cells were co-cultured for 24-72 h. Death of CT26 cells was determined by the flow cytometry. Results are presented as mean±SD (n=2). (B) The effect of treatment with PD-L1ip3 in peptide form and transduction of Ad-PD-L1ip3 on death of CT26 cells induced by antigen-primed T cells. CT26 cells treated with PD-L1ip3 in peptide form (10 μM) or transduced by Ad-PD-L1ip3 (100 MOI) was cocultured with antigen-primed T cells at a 1:16 ratio. Cell death of CT26 was evaluated as same with (A). Anti-PD-L1 antibody (αPD-L1; 0.5 μg/ml) was used as a positive control. PBS was used as a negative control. Results are presented as mean±SD (n=2).

FIG. 5 is a graph showing treatment with Ad-PD-L1ip3 transduction alone or in combination with PD-L1ip3 in peptide form attenuated the growth of subcutaneously inoculated CT26 cell tumors in mouse. The vector carrying the scrambled peptide gene (AD-PD-L1ip3SC) did not show any significant tumor growth attenuation as compared to untreated CT26 cell tumors. Results are presented as mean±SD (n=5).

FIG. 6 shows graphs of data indicating that prior stimulation of host immunity by CT26 cell lysate enhanced the inhibitory effect of PD-L1ip3 and attenuated the growth of subcutaneously inoculated CT26 tumors in mouse by apoptosis. (A-E) Panel A shows average tumor size, while panels B-E show volumes of individual tumors (dotted lines) in each treatment group. Solid line in panels B-E show average tumor size in each group. Results are presented as mean±SD (n=5-6). *; P<0.05 with Naïve CT26, ⋄; P<0.05 with PD-L1ip3 peptide (F) Treatment with Ad-PD-L1ip3 transduction and combination with PD-L1ip3 peptide treatment increased apoptotic cells in CT26 tumors. Results are presented as mean±SD (n=5).

FIG. 7 shows a graph from Example 2 of data for death fold change in cell populations subjected to various treatments.

FIG. 8 is a graph showing the data for cell death in murine lung cancer cells subjected to various treatments in Example 3.

FIG. 9 is a graph showing the significant increase in survival of mice in a mouse cancer model after treating with the adenovirus vector encoding the inhibitory peptide (Ad-PD-L1ip3) as compared to the control (CNT) from Example 4.

FIG. 10 is a graph showing the significant increase and duration of inhibitor peptide expression from the vaccinia viral vector-infected CT26 cells.

FIG. 11 is a graph showing the significant decrease in tumor size (by weight) after treatment with vaccinia viral vector encoding the inhibitory peptide (VV-PD-L1ip3) as compared to a control (CNT).

### DETAILED DESCRIPTION

PD-L1 molecules expressed on cancer cells attenuate cytotoxic T cell activities against cancer cells by interacting with PD-1 molecules expressed on T cells and downregulating an immune response against the cancer. Disclosed herein is a novel PD-L1 inhibitory peptide designed for high binding specificity for human PD-L1.

The PD-L1 inhibitory peptide (PD-L1ip3) blocks the interaction of PD-L1 with PD-1 molecules on T cells, and therefore interrupts the attenuation of cytotoxic T cell function by immune checkpoint mechanisms thus triggering the immune response against cancer cells in the patient. The terms “cancer” and “tumor” are used interchangeably herein to refer to an abnormal cell or population of cells (tissues) characterized by uncontrolled growth and/or proliferation and which sometimes spread to other parts of the body (metastasis). Cancer cells typically display tumor antigens.

In one or more embodiments, the present disclosure concerns therapeutic peptides that can be used to enhance immune responses against cancer cells, including enhancing a local immune response in cancer tissue, or enhance effectiveness of a cancer treatment by administration of the peptides as immune checkpoint inhibitors. The inhibitory peptide comprises the sequence:

PD-L1ip3:
(SEQ ID NO: 1)
GTRLKPLIICVQWPGL

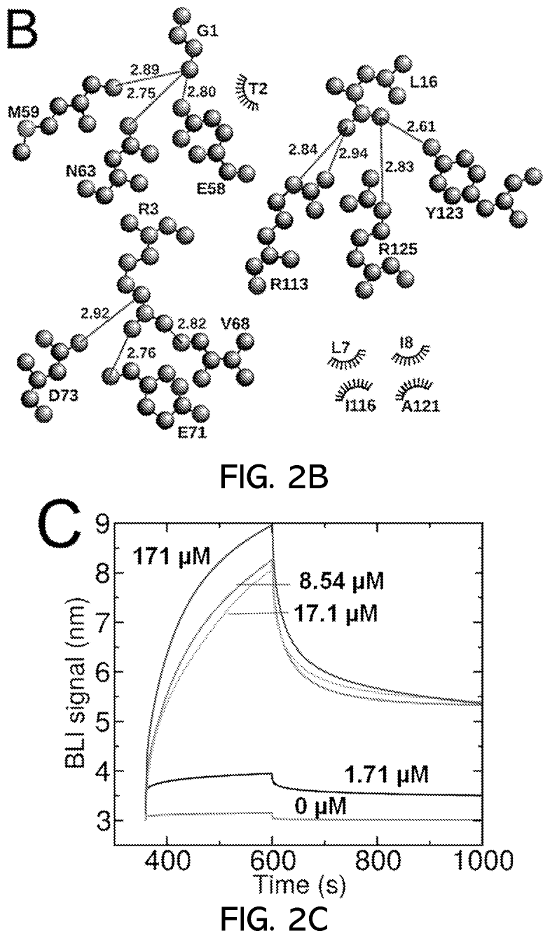

The inhibitory sequence of the peptide is a short, linear oligopeptide sequence of less than 20 amino acids. As shown in the working examples, the binding affinity of PD-L1ip3 for human PD-L1 measured by bio-layer Interferometry revealed that PD-L1ip3 binds to human PD-L1 with a dissociation constant of KD=33±3 μmol/L (FIG. 2), which is only a few times weaker than the binding of PD-L1 to its natural partner PD-1 (KD≈8 μmol/L). It should be noted that human PD-L1 shows a similar affinity for murine PD-1 as for human PD-1, indicating the binding affinity is highly conserved.

Peptides can be prepared by various synthetic methods of peptide synthesis, such as via condensation of one or more amino acid residues, in accordance with conventional peptide synthesis methods. Synthesis can also be carried out using standard solid-phase methodologies, such as may be performed on a commercial automatic synthesizer. Other methods of synthesizing peptides, either by solid-phase methodologies or in liquid phase, are well known to those skilled in the art and are contemplated herein, such as by Boc-mediated solid-phase peptide synthesis or Fmoc-based chemistries.

In one or more embodiments, the peptides can also be obtained from polynucleotides encoding the peptide and expressed in vivo or ex vivo for treatment. The polynucleotide may be comprised in or on a vector. The vector may or may not be oncolytic. Any suitable expression vector can be used, i.e., a vector that contains the necessary elements for the transcription and translation of the inserted peptide coding sequence in the host cell, such as control elements to direct or initiate expression of the inserted peptide coding sequence, including those involved in regulating expression or facilitating of translation in a target host cell, as well as those involved in replication and/or assembly of viral particles in infected cells, and/or integration into the host genome. Any viral or non-viral vector may be used in vivo or ex vivo to deliver the polynucleotides into cancer cells and/or cells within the cancer microenvironment. For example, viral vectors containing coding sequences encoding the inhibitor peptide can be prepared. Suitable viral vectors include retrovirus vectors, adenoviral vectors, vaccinia virus vectors, lentivirus, adeno-associated virus, and the like. Recombinant viral vectors generally comprise a single-stranded DNA molecule comprising the inserted heterologous sequence flanked by viral and/or non-viral regulatory sequences. Recombinant viral vectors can be encapsulated into virus particles to form recombinant viruses. Both the recombinant viral vectors and virus particles containing the recombinant viral vectors can be used to express the inhibitory peptides in host cells by transformation or transduction, respectively.

A nucleic acid sequence encoding the inhibitory peptide (the expression cassette) can be incorporated into a nonessential region of the virus genome, that is, an insertion region that does not affect viability of the resultant recombinant virus. Many such regions for common viral vectors are known. One of skill in the art can also readily identify such regions by testing (e.g., in silico) segments of the viral genome for regions that allow recombinant formation and packaging of the viral structure without significant impact on viability of the recombinant virus. Commercially available kits for preparing viral vectors are also available, along with custom vector synthesis services. Non-viral vectors for use in the invention include nanoparticles, lipids, lipid polymers, and the like.

The nucleic acid sequence encoding the inhibitory peptide is operably linked to regulatory or expression control sequence(s) that drive expression of the nucleic acid encoding the peptide and allow the polynucleotide to be expressed in any cell in which it resides (infects) and become secreted so that it may act upon its target (i.e., PD-L1). Thus, host cells and virus particles comprising the recombinant viral vectors of the invention are also provided. Regulatory sequences include promoters, leader sequences, signal sequences, etc. Epitope tags for detecting the expression of recombinant peptides can also be included in the nucleic acid constructs, along with linkers, etc. as exemplified in the working examples. In some embodiments, the nucleic acid sequence encoding the inhibitory peptide is transfected into cells ex vivo and the cells harboring the polynucleotide are locally delivered to a tumor or into the cancer microenvironment such that the manipulated cells produce the inhibitory peptide and then secrete the inhibitory peptide into the local site so that it can be bind to its target (i.e., PD-L1 expressed on cancer cells in the site). In some embodiments, a viral vector comprising a nucleic acid sequence encoding the inhibitory peptide is locally delivered to a tumor or into the cancer microenvironment.

Thus, contemplated herein are nucleic acids encoding the inhibitory peptide, vectors including these nucleic acids, host cells including these nucleic acids, and immunogenic compositions including the inhibitory peptide, nucleic acids and/or host cells are also disclosed. Pharmaceutical compositions including a therapeutically effective amount of the inhibitory peptide, nucleic acids, and/or host cells are also described. As such, the methods include administering to the subject a therapeutically effective amount of the inhibitory peptide or a nucleic acid sequence encoding the inhibitory peptide.

Regardless of the embodiment, the peptide has an affinity and specificity for binding PD-L1, which is expressed on cancer cells, and in doing so facilitates an immune response against the cancer cells by blocking the downregulation of immune cell function that would be induced if the PD-L1 remained free to bind to PD-1. As used herein, an “immune response” refers to the action, stimulation, or activation of the cells of the immune system, including lymphocytes (T and B cells), natural killer (NK) cells, and macrophages, which normally react and respond to foreign organisms, damaged or diseased/abnormal cells, etc. which results in selective damage to, destruction of, or elimination from the human body of these foreign or “non-self” substances. Thus, the immune response can be detected directly, such as by an increase in immune cell activity, anti-tumor or activated T cell numbers, or secretion products, or indirectly such as by reduction in tumor size (tumor shrinkage), decreased rate of growth (tumor regression), stalled growth (tumor stabilization), prolonged survival, reduced pain at the tumor site, extension of lifespan, improvement in quality of life, and the like, which are attributable to clinically relevant improvement in one or more markers of disease status and progression (as compared to the subject prior to treatment).

In one or more embodiments, analogs or derivatives of SEQ ID NO:1 are contemplated herein. Analogs or derivatives may comprise conserved amino acid substitutions (preferably 2 or less) and or side chain functionalization. In one or more embodiments, the peptide may comprise a functional moiety or label may be attached to a side chain of any one or more of residues, such as Va11, Gln12, or Trp13. In one or more embodiments, the peptide may comprise a detectable moiety such as a fluorescent dye or peptide. Fluorescently labeled peptides can be used for cell culture (microscopic observation), flow cytometry, and histological observation (e.g., with confocal microscope). Peptide modifications are known in the art, including N-terminal (e.g., biotin, 5-FAM, Abz, Boc, CBZ, Fmoc, and the like) or C-terminal modifications (e.g., AFC, AMC, Amidation, Esterification, and the like), stable isotope labels (e.g., Ile(13C6,15N), Lys(13C6,15N2), Val(13C5,15N), and the like), fluorescent moieties (5-FAM, Abz, DABCYL, Fluorescein isothiocyanate (FITC), MCA, and the like).

In one or more embodiments, the peptides can be modified or functionalized for targeting cancer tissues, such as by attaching a targeting moiety or ligand having affinity for cancer cells, such as RGD peptides which preferentially accumulate near cancer tissue. It will also be appreciated that the affinity of the peptides for PD-L1 provides the peptides with a cancer targeting ability. Thus, in some embodiments, the peptides may themselves be considered tumor-targeting moieties that could be attached to other active agents.

For therapeutic uses, the inhibitory peptide or a nucleic acid sequence encoding the inhibitory peptide are typically administered as part of a composition comprising a plurality of the inhibitory peptides or a nucleic acid sequences encoding the inhibitory peptides dispersed in a pharmaceutically-acceptable carrier. The term carrier is used herein to refer to diluents, excipients, vehicles, and the like, in which the inhibitory peptide or nucleic acid sequence encoding the inhibitory peptide may be dispersed for administration. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would be selected to minimize any degradation of the peptide or other agents and to minimize any adverse side effects in the subject. Pharmaceutically-acceptable ingredients include those acceptable for veterinary use as well as human pharmaceutical use and will depend on the route of administration. For example, compositions suitable for administration via injection are typically solutions in sterile isotonic aqueous buffer. Exemplary carriers include aqueous solutions such as normal (n.) saline (˜0.9% NaCl), phosphate buffered saline (PBS), sterile water/distilled autoclaved water (DAW), various oil-in-water or water-in-oil emulsions, as well as dimethyl sulfoxide (DMSO), other acceptable vehicles, and the like.

The composition can comprise a therapeutically effective amount of the inhibitory peptide or nucleic acid sequence encoding the inhibitory peptide dispersed in the carrier. As used herein, a “therapeutically effective” amount refers to the amount that will elicit the biological or medical response of a tissue, system, or subject that is being sought by a researcher or clinician, and in particular elicit some desired therapeutic effect as against the cancer cells by blocking the interaction of PD-L1 with PD-1, allowing the CTLs to be activated. Thus, the inhibitory peptide or nucleic acid sequence encoding the inhibitory peptide are preferably provided in an amount sufficient to block a suitable quantity of PD-L1 (or suitable quantity of cancer cells) to facilitate activation of an effective amount of immune cells against the cancer cells. One of skill in the art recognizes that an amount may be considered therapeutically “effective” even if the condition is not totally eradicated or prevented, but it or its symptoms and/or effects are improved or alleviated partially in the subject, such as reduction in number/volume of cancer cells or tissue/tumor nodules and/or reduction in rate of growth of the cancer cells, or tissue/nodules even if the cancer is not totally eradicated. That is, the immune checkpoint inhibitor peptides can be useful in enhancing the immune response against the cancer and reducing or stalling the cancer, such that adjunct therapies such as chemotherapy or other immunotherapy, and the like can have a greater impact on thereafter eradicating the cancer cells. Thus, it is contemplated that the immune checkpoint inhibitor peptides may be used as part of a multi-faceted cancer treatment plan. In some embodiments, the composition will comprise from about 5% to about 95% by weight of the peptides described herein, and preferably from about 30% to about 90% by weight of the peptides, based upon the total weight of the composition taken as 100% by weight. Encapsulation techniques can also be used to facilitate delivery of the peptides.

Other ingredients may be included in the composition, such as adjuvants, other active agents (e.g., other checkpoint inhibitors, immunotherapies, chemotherapies), preservatives, buffering agents, salts, other pharmaceutically-acceptable ingredients. The term “adjuvant” is used herein to refer to substances that have immunopotentiation effects and are added to or co-formulated in a therapeutic composition in order to enhance, elicit, and/or modulate the innate, humoral, and/or cell-mediated immune response against the active ingredients. Other active agents that could be included in the composition include any immunogenic active components (e.g., monoclonal antibodies, tumor antigens, etc.), such that it provokes a general increase in immune response in the patient and/or a targeted immune response against the cancer cells.

In use, a therapeutically-effective amount of the inhibitory peptide or nucleic acid sequence encoding the inhibitory peptide is administered to a subject. In some embodiments, a composition comprising a therapeutically-effective amount of the inhibitory peptide or nucleic acid sequence encoding the inhibitory peptide is administered to a subject. The disclosed embodiments are suitable for various routes of administration, depending upon the particular carrier and other ingredients used. For example, the peptides can be injected intramuscularly, intraperitoneally, subcutaneously, intradermally, intratumorally, intratracheally, or intravenously. They can also be administered via mucosa such as intranasally or orally. The compounds or compositions can also be administered through the skin via a transdermal patch, or topically applied to dermal and epidermal-based cancers (e.g., melanoma). In one or more embodiments, the inhibitory peptide or nucleic acid sequence encoding the inhibitory peptide is formulated for intratumoral administration, wherein the peptide or nucleic acid is locally administered in or near the site of cancer cells or a tumor. Intratumoral administration of the peptide results in changes in the tumor microenvironment, including binding PD-L1 and facilitating activation of the immune system. Nucleic acid sequences encoding the inhibitory peptide (and vectors carrying the same) can also be delivered using the above methods, as well as by gene gun, electroporation, hydrodynamic, ultrasound, and the like. In preferred embodiments, the inhibitory peptide or nucleic acid sequence encoding the inhibitory peptide is administered locally (and not systemically) at the cancer or tumor site or microenvironment.

As noted, the inhibitory peptide or nucleic acid sequence encoding the inhibitory peptide can be administered alone or co-administered with other immunotherapies and/or chemotherapies. In one or more embodiments, coadministration means simultaneous administration of two or more active agents, either in the same composition or at the same time but in respective compositions (e.g., separate injections, or into the same IV drip line, etc.). Coadministration may also refer to sequential administration of the active agents, separated by minutes or hours, but typically within the same day (24 hour period). In one or more embodiments, the checkpoint inhibitor peptides or nucleic acid sequence encoding the inhibitory peptides may be administered as part of a multi-faceted cancer treatment program for a subject.

In one or more embodiments, the inhibitory peptide itself is co-administered with a nucleic acid sequence separately encoding the inhibitory peptide. That is, in one or more embodiments, the treatment comprises directly administering an effective amount of the inhibitory peptide itself (or composition containing a plurality of inhibitor peptides) to the subject. The subject is also administered a nucleic acid sequence encoding the inhibitory peptide, and preferably a vector, more preferably a viral vector comprising the nucleic acid sequence encoding the inhibitory peptide. As demonstrated in the working examples, this combined treatment approach achieves an enhanced effect in reducing tumor size and volume, inhibiting cancer cell activity, and improving the immune response against the cancer cells in the treated site. Without wishing to be bound by theory, the direct treatment with the peptides themselves leads to initial binding and interference with PD-L1 on cancer cells allowing the CTLs to become more activated, while delayed and ongoing expression/secretion of the peptides from the administered nucleic acids also administered to the site reinforces and maintains the blocking of PD-L1 and concomitant stimulation of the immune response in the site, allowing for a sustained immunotherapy response (more than 24 hours after administration, preferably more than 72 hours). Thus, the combined approach can lead to an enhanced and even synergistic anti-tumor immunity compared to either treatment alone. As noted, the approach is effective for reducing tumor size, volume, cancer cell activity, and enhancing the immune response, particularly for lung cancers and colorectal cancers.

In one or more embodiments, the checkpoint inhibitor peptides or nucleic acid sequence encoding the inhibitory peptides may be used as the primary cancer treatment. In one or more embodiments, the checkpoint inhibitor peptides or nucleic acid sequence encoding the inhibitory peptides may be used as an adjunctive treatment. Adjunctive treatments are typically those given after the primary treatment to lower the risk that the cancer will come back. Additional adjunctive treatments that may be administered alongside of the checkpoint inhibitor peptides or nucleic acid sequence encoding the inhibitory peptides may include chemotherapy, radiation therapy, hormone therapy, or other immunotherapy. Those skilled in the art can develop the appropriate treatment plan based upon the particular cancer involved, the stage of the cancer, prognosis, and age of the patient.

Upon administration, the checkpoint inhibitor peptides bind to and block PD-L1 and prevent its binding PD-1 on CTLs allowing the immune system to detect and react to the cancer cells leading to an immune response, typically characterized by cytotoxic T cell-dependent oncolysis. Embodiments described herein thus include methods of enhancing an immune response to cancer cells. Also described herein are methods of facilitating activation of an immune cell at a cancer site comprising cancer cells.

In some embodiments, the checkpoint inhibitor peptides, nucleic acids encoding the inhibitor peptides, or compositions thereof can be provided in unit dosage form in a suitable container. The term “unit dosage form” refers to a physically discrete unit suitable as a unitary dosage for human or animal use. Each unit dosage form may contain a predetermined amount of the checkpoint inhibitor peptides, nucleic acids encoding the inhibitor peptides, or compositions thereof (and/or other active agents) in the carrier calculated to produce a desired effect. In other embodiments, the checkpoint inhibitor peptides or nucleic acid sequence encoding the inhibitory peptides can be provided separate from the carrier (e.g., in its own vial, ampule, sachet, or other suitable container) for on-site mixing before administration to a subject. A kit comprising the checkpoint inhibitor peptides or nucleic acid sequence encoding the inhibitory peptides is also disclosed herein. The kit further comprises instructions for administering the checkpoint inhibitor peptides or nucleic acid sequence encoding the inhibitory peptides to a subject. The checkpoint inhibitor peptides or nucleic acid sequence encoding the inhibitory peptides can each be provided as part of a dosage unit, already dispersed in a pharmaceutically-acceptable carrier, or provided separately from the carrier. The kit can further comprise instructions for preparing the checkpoint inhibitor peptides or nucleic acid sequence encoding the inhibitory peptides for administration to a subject, including for example, instructions for dispersing the checkpoint inhibitor peptides or nucleic acid sequence encoding the inhibitory peptides in a suitable carrier.

It will be appreciated that therapeutic and prophylactic methods described herein are applicable to humans as well as for veterinary use for any suitable animal, including, without limitation, dogs, cats, and other companion animals, as well as, rodents, primates, horses, cattle, pigs, etc. The methods can be also applied for clinical research and/or study.

As demonstrated below, the biological activity of the inhibitory peptides is confirmed in cell culture and in mouse. Bioactivity has also been evaluated using peptide form of the inhibitory peptides, as well as in an adenovirus vector encoding for the peptide, and vaccinia virus vector encoding for the peptide. The peptide has been validated experimentally for its specific binding to PD-L1, and evaluated for its antitumor effects in cell culture and in a mouse colon carcinoma allograft model. In cell culture studies, direct treatment with PD-L1ip3 substantially increased death of CT26 colon carcinoma cells when co-cultured with murine CD8⁺ T cells primed by CT26 cell antigens. In a mouse allograft tumor model, the growth of CT26 tumor cells transduced with the PD-L1ip3 gene by an adenovirus vector was significantly slower than that of un-transduced CT26 cells in immunocompetent mice. This tumor growth attenuation was further enhanced by coadministration of the peptide form of PD-L1ip3 (10 mg/kg/day). This study suggests that this peptide is capable of stimulating host antitumor immunity via blockade of the PD-1/PD-L1 pathway, thereby increasing CD8⁺ T cell-induced death of cancer cells. The tumor site-specific inhibition of PD-L1 by an adenovirus carrying the PD-L1ip3 gene, together with direct peptide treatment, may be usable as a local immune checkpoint blockade therapy that can inhibit cancer.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Peptide Design and In Vitro and In Vivo Validation Against Colon Cancer

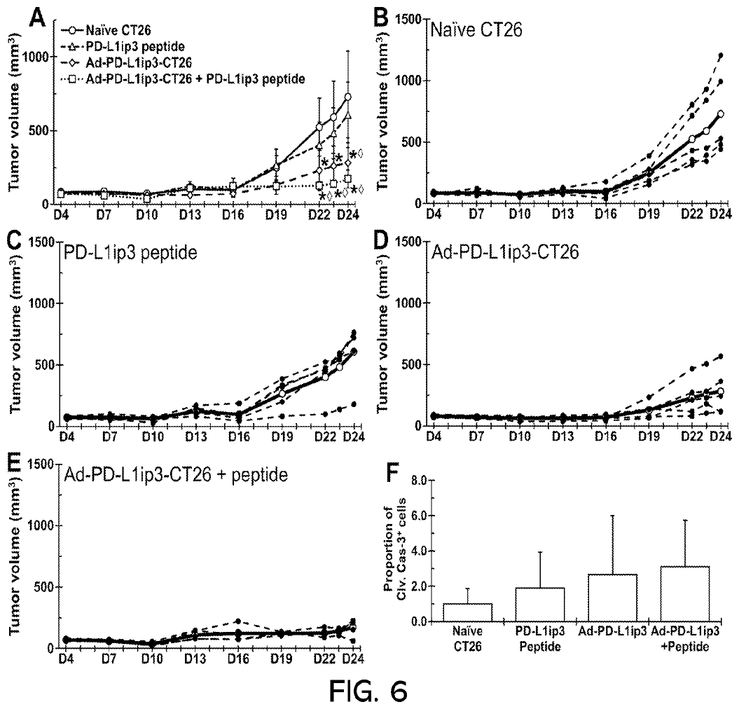

In this work, novel inhibitory peptide, PD-L1ip3, interfering PD-1/PD-L1 immune checkpoint pathway was designed. Affinity of PD-L1ip3 to PD-L1 was only a few times weaker than that of the natural ligand, PD-L1. Treatment of PD-L1ip3 enhanced cytotoxicity of cancer antigen specific CD8⁺ T cell against cancer cells in cell culture. Combination treatment of PD-L1ip3 gene expression and its peptide form significantly attenuated the growth of murine colon carcinoma in mice. Ad-PD-L1ip3 transduction (100 MOI) into CT26 cells significantly attenuated the growth of the tumors (281.4±169.6 mm³) compared to the untreated CT26 cell tumors (729.6±310.3 mm³). The effect of Ad-PD-L1ip3 transduction was further enhanced by the combination treatment with PD-L1ip3 peptide (10 mg/kg/day, intraperitoneal injection) (172.6±60.9 mm³). On the other hand, the treatment with PD-L1ip3 peptide alone showed only minimal effect on the tumor growth (607.5±220.1 mm³) (FIG. 6). Immunohistochemical analysis of apoptotic marker (cleaved caspase-3) in tumor demonstrated that the tumor attenuation by PD-L1ip3 gene transduction and treatment with PD-L1ip3 peptide was caused by induction of apoptosis in cancer cells (FIG. 6). These results suggest that our PD-L1 blockade therapy by adenoviral vector-based PD-L1ip3 gene therapy along with the treatment with the PD-L1ip3 peptide is effective in inhibiting tumor growth. The combination treatment with the PD-L1ip3 gene transduction in tumor cells and the inhibitory peptide almost completely inhibited the growth of tumor presumably due to the blockade of PD-L1 and PD-1immune checkpoint mechanism.

It was observed in the differences on the effect of PD-L1ip3 peptide and Ad-PD-L1ip3 transduction between in cell culture and mouse study. One of reasons is what it was not enough time to express PD-L1ip3 gene in CT26 cells. In cell culture study, the effect of PD-L1ip3 gene on cytotoxicity of CT26-CTL was evaluated within 72 hours after gene transduction, but within 20 days after gene transduction in mouse study. Another reason is what the effect of PD-L1ip3 gene transduction on the growth of CT26 tumor was evaluated by local treatment for tumor using direct transduction of gene into CT26 cells.

### INTRODUCTION

We report on the design of a novel peptide that specifically binds PD-L1 and experimental results demonstrating that a combination treatment of an adenovirus vector encoding a secretory gene for this peptide along with direct administration of the peptide itself inhibits the growth of murine colon carcinoma cells in cell culture and in a mouse allograft model via a stimulation of cytotoxic T cell function.

### Materials and Methods

### Animals

Female Balb/c mice were obtained from Charles River Laboratories International, Inc. All mice were housed in a clean facility and acclimatized for 7 days. All animal experiments adhered strictly to protocols approved by the Kansas State University Institutional Animal Care and Use Committee (Protocol #4393) and Institutional Biosafety Committee (Protocol #1317).

### Materials

The mouse colon carcinoma cell line CT26.CL25 (CRL-2639) and mouse immature dendritic cell line JAWSII (CRL-11904) were purchased from American Type Culture Collection (ATCC, Manassas, VA). RPMI 1640 was from Mediatech, Inc. (Manassas, VA). Fetal bovine serum (FBS) was from Biowest (Riverside, MO). Penicillin-streptomycin stock was obtained from Lonza Rockland, Inc. (Allendale, NJ). 200 mM sodium pyruvate, MEM non-essential amino acids (100×), MEM amino acids (100×), 200 mM L-glutamine, antibiotic-antimycotic (50×) (all are Gibco®) were from Thermo Fisher Scientific (Waltham, MA). 2-mercaptoethanol was purchased from Sigma-Aldrich (St. Louis, MO). Anti-cleaved Caspase-3 antibody (Asp175) was from Cell Signaling Technology, Inc. (Danvers, MA).

### Bio-Layer Interferometry

Binding of the computationally designed PD-L1ip3 peptide to PD-L1 was verified using a FortéBio BLItz machine (Freemont, CA, USA). The PD-L1ip3 peptide and its scrambled analogue were synthesized by GenScript (Piscataway, NJ) with no modifications or labels. Biotinylated human PD-L1 and untagged PD-1 (a positive control) were purchased from R&D Systems (Minneapolis, Minnesota). For each experiment, the BLItz machine was fitted with a fresh streptavidin biosensor tip (FortéBio). All tips were solvated in PBS buffer for 15-30 min before the experiments. The peptide was solvated at a concentration of approximately 1 mg/ml. The precise concentration of this stock solution (0.922 mg/ml) was obtained using the Pierce BCA Protein Assay Kit (Thermo Fisher Scientific) and multiple dilutions were made (corresponding to 514, 171, 17.1, 8.54, and 1.71 M). A solution of biotinylated PD-L1 was applied to the biosensor tip during the loading phase of each experiment. PBS buffer (ix) was used for the baseline. The scrambled-sequence peptide, PD-L1ip3SC, served as the negative control and PD-1, the natural binding partner of PD-L1, was the positive control.

### Design of PD-L1 Inhibitory Peptide

To generate candidate PD-L1 binding peptides, the PinaColada program was applied to an x-ray structure of human PD-1 bound to human PD-L1 (PDB ID: 4ZQK) as a template. We selected 14 candidate sequences from the output of the algorithm, which were then docked into PD-L1 near the PD-L1:PD-1 binding interface using the CABS-dock server for flexible protein-peptide docking. We obtained 6 docked complexes for each sequence. The docked complexes were then simulated for 10 ns in explicit solvent molecular dynamics using the program Amber16. We estimated binding free energies by the MM-PBSA method (molecular mechanics, Poisson-Boltzmann and surface area), as implemented in Amber16. The MM-PBSA estimates of the binding free energies for the 14 sequences are given in Table 1. All molecular dynamics simulations used the Amber ff12SB force field, a timestep of 2 fs, the particle-mesh Ewald method for full electrostatics, and a cutoff of 9 Å for explicit non-bonded interactions. The temperature was maintained at 300 K using a Langevin thermostat and the pressure was maintained at 1 atm using the Monte Carlo barostat (Amber16) or Langevin piston barostat (NAMD). Long simulations of the peptides in contact with PD-L1 were performed using NAMD 2.12.

TABLE 1
Estimated binding free energies (in kcal/mol) for
peptide binding to PD-L1 calculated by MM-PBSA.

| | ΔG_{MM-PBSA} | SEQ |
|---|---|---|
| Candidate Sequences | (kcal/mol) | ID NO: |
| AISLHPKAKILEWPGA | −47.5 | 2 |
| PLDIRDRVHVEKSAAS | −56.5 | 3 |
| (PD-L1ip4) | | |
| SVAVNPTPTLMDAPGG | −30.9 | 4 |
| SVAVNDTPTLMDAAAG | −32.5 | 5 |
| FHTLEPSLLAINTPGV | −31.4 | 6 |
| FHTIEDSLLAINTAAV | −31.9 | 7 |
| FHTVEPSLLAINTPGV | −32.5 | 8 |
| GTRLKPLIICVQWPGL | −58.7 | 1 |
| (PD-L1ip3) | | |
| GTRIKDLIICVQSAAL | −53.6 | 9 |
| GTRVKPLIICVQAPGL | −43.5 | 10 |
| LIELHPAARITEWPGA | −44.7 | 11 |
| LLEIRDAVRVTKSAAS | −38.5 | 12 |
| VISLHDKAAIHEWPGA | −46.1 | 13 |
| VLDIRPRVA VHKSAAA | −39.6 | 14 |

### Preparation of Adenovirus Vector

The present example exemplifies one way to construct a viral vector, but should not be construed as limiting. To generate an adenovirus expression vector encoding a gene for secreting PD-L1ip, an adenovirus expression plasmid was constructed using the pENTR/D-TOPO plasmid (the entry vector) and the Gateway-based pAd/CMV/V5-DEST vector (the destination vector) (Invitrogen Corp, Carlsbad, CA). The DNA sequence encoding an N-terminal secretion signal (METDTLLLWVLLLWVPGSTGD, SEQ ID NO:15) from the V-J2-C region of the mouse Ig kappa-chain, a short linker (AAQPARRA, SEQ ID NO:16), a c-Myc(-L) tag (EQKLISEED (SEQ ID NO:17), i.e., Myc tag EQKLISEEDL (SEQ ID NO:18), without L), and the PD-L1ip3 sequence (GTRLKPLIICVQWPGL, SEQ ID NO:1) was synthesized and was cloned into the entry vector. The whole amino acid sequence contains a signal peptide (21 amino acids)+linker peptide (8 amino acids)+c-Myc tag peptide without C-terminus L (9 amino acids)+PD-L1ip3 peptide (16 amino acids):

(SEQ ID NO: 19)
METDTLLLWVLLLWVPGSTGD-AAQPARRA-EQKLISEED-
GTRLKPLIICVQWPGL

### Nucleotide Sequence:

(SEQ ID NO: 20)
ATGGAAACTGATACTCTACTACTATGGGTTCTACTACTATGGGTTC
CAGGTAGTACTGGTGATGCAGCACAACCTGCAAGAAGAGCAGGTAC
CCGTCTAAAACCACTAATTATTTGTGTTCAATGGCCAGGTCTA

It will be appreciated that the signal peptide is not included in the final secretory peptide (e.g., AAQPARRA-EQKLISEED-GTRLKPLIICVQWPGL, SEQ ID NO:21) since the signal peptide is cleaved between the signal peptide and the linker peptide when this gene product is secreted out from the infected cells. It will also be appreciated that the expression vectors are not limited to the particular nucleotide sequences shown above, as other suitable DNA and/or mRNA sequences could be designed having sequence variations which would nonetheless still encode for the desired final secretory peptide.

A version with a scrambled PD-L1lip3 sequence (VLIKIPWLRLPGCGTQ, SEQ ID NO:22) was also synthesized and cloned into an entry vector. The scrambled expression vector (Ad-PD-L1ip3SC) contains nucleotide sequences encoding for the same signal peptide sequence, linker peptide, and c-Myc(-L) tag as above.

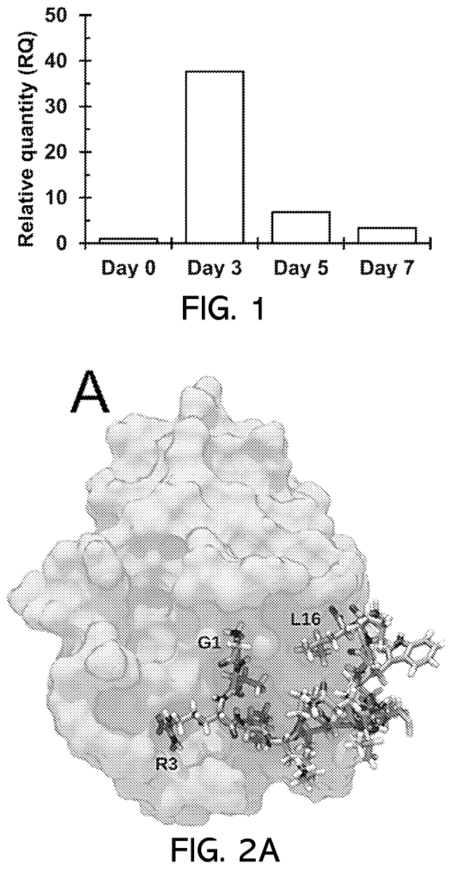

The inserts were then transferred from the entry vector into the destination vector by the LR recombination reaction according to the manufacturer's instructions. The destination vectors thus obtained were digested by Pac I and were transfected into 293A cells to produce crude adenoviral vector stocks. The adenovirus vectors were amplified by infecting 293A cells. DNA sequence analysis of adenovirus vectors for PD-L1ip3 (Ad-PD-L1ip3) and for the scrambled version (Ad-PD-L1ip3SC) revealed that the sequences of the inserts were correct. Since Ad-PD-L1ip3 produces a larger size polypeptide (SEQ ID NO:21) than the original oligopeptide form of PD-L1ip3 (SEQ ID NO:1) due to the addition of the linker (SEQ ID NO:16) and c-Myc tag sequences (SEQ ID NO:12) in the final secretory peptide, additional MD simulations were performed to verify that the complete secreted peptide constructs also led to stable complexes. Indeed, a slightly greater affinity was predicted when the linker and c-Myc tag was included. PD-L1ip3 gene expression in Ad-PD-L1ip3 transduced CT26 cells was confirmed by RT-qPCR as described in a previous paper, using a primer pair with the forward primer 5′-TATCAGCGAGGAGGACGGTA-3′ (SEQ ID NO:23) and reverse primer 5′-CAGATGATCAGGGGCTTCAG-3′ (SEQ ID NO:24) (FIG. 1).

### Cell Culture

The CT26 murine colon carcinoma cells were cultured in RPMI 1640 supplemented with 10% v/v FBS and 1% v/v penicillin-streptomycin. Murine CD8⁺ T cells were cultured in RPMI 1640 supplemented with 1 mM sodium pyruvate, lx MEM non-essential amino acids, lx MEM amino acids, 1 mM L-glutamine, 1× antibiotic-antimycotic and 0.36 mM 2-mercaptoethanol. These cells were cultured at 37° C. in a humidified air atmosphere containing 5% CO₂. Cell line was authenticated by short-tandem repeat (STR) DNA profiling and maintained in low passage (<15) for this study.

Evaluation of the Effect of PD-L1ip3 Peptide or Gene Transduction by Ad-PD-L1ip3 on Murine Colon Carcinoma Cells in Cell Culture The CT26 murine colon carcinoma cells (1,000 cells/well) were seeded into 96-well plate.

After 24 h, these cells were treated directly with PD-L1ip3 in peptide form (0.1-10 μg/ml) or indirectly with the PD-L1ip3 gene by Ad-PD-L1ip3 transduction (10, 20 and 50 MOI). The growth of the cells was evaluated by MTT assay 24-72 h after the treatments as described previously.

Evaluation of the Effect of PD-L1ip3 Peptide or Gene Transduction by Ad-PD-L1ip3 on T Cell-Induced Death of Murine Colon Carcinoma Cells in Co-Culture with Antigen-Primed T Cells

a. Generation of CT26 cell antigen-specific CD8⁺ T cells in vivo: First, the mice were subcutaneously (SQ) injected with CT26 cell lysate (0.5×10⁶ cells/mouse in 200 μl PBS) at Day 0. JAWSII immature dendritic cells and CT26 cells previously irradiated with an X-ray dose of 100 Gy were cocultured at a 1:1 ratio for 48 h with an additional treatment of LPS (1 μg/ml). The cells (0.5×10⁶ cells/mouse in 200 μl PBS) were collected and administered IV via tail vein to the mice at Day 7. At Day 21, the splenocytes were harvested and the CT26 antigen-specific CD8⁺ T cells were labeled using MojoSort™ Mouse CD8 T Cell Isolation Kit (Biolegend, San Diego, CA) and isolated using magnetic beads-based separation protocol with MACS® Column (Miltenyi Biotec, Bergisch Gladbach, Germany) according to the manufacturer's instructions.

b. Evaluation of T cell-induced death of colon carcinoma cells in co-culture: Permanently GFP-expressing CT26 cells (GFP-CT26) produced by GFP-lentivirus vector transduction were seeded into 12 well-plate (1×10⁴ cells/well) in the presence of murine interferon gamma (mIFNγ) at 25 ng/ml for 24 h. The cells were treated directly by PD-L1ip3 peptide (10 μM) or indirectly by Ad-PD-L1ip3 transduction (100 MOI) treated 30 min and 24 h before coculturing with CD8⁺ T cells (1:16 ratio), respectively. The effect of PD-L1ip3 in peptide form or gene transduction by Ad-PD-L1ip3 on CT26 cell death was analyzed by flow cytometry using LIVE/DEAD™ Fixable Violet Dead Cell Stain Kit (Invitrogen Corp). The specific death of GFP-CT26 cells was identified by GFP⁺ LIVE/DEAD⁺ gating. The Ultra-LEAF™ Purified anti-mouse CD274 (B7-H1, PD-L1) antibody (0.5 μg/ml; BioLegend) was used as positive control of interruption of PD-1/PD-L1 interaction. PBS, PD-L1ip3SC peptide and Ad-PD-L1ip3SC were used as negative controls.

Evaluation of Ad-PD-L1ip3 Treatment and Cotreatment with PD-L1ip3 Peptide on Colon Carcinoma Tumor Growth in a Subcutaneous Allograft Tumor Model

The anti-tumor effect of Ad-PD-L1ip3 and PD-L1ip3 in peptide form was evaluated in Balb/c mice using a CT26 murine colon carcinoma allograft model. The CT26 cells were transduced with 100 MOI of Ad-PD-L1ip3 or Ad-PD-L1ip3SC. At 24 h after transduction, the cells were suspended into 0.6% agarose dissolved in RPMI 1640. After filling agarose-cell mixture into 1 ml syringe equipped with 27G needle, it was solidified on ice. Mice were anesthetized with isoflurane and injected subcutaneously into the back with a 5×10⁵ CT26 cells in 50 μl cell-agarose mixture. The intraperitoneal administration of PD-L1ip3 peptide (10 mg/kg) was carried out at 1 week after CT26 inoculation for 13 days (every day, totaling 13 injections). The PBS control was injected intraperitoneally with the same schedule. For the pre-immunization study, CT26 lysate (1×10⁶ cells/100 μl PBS) were injected subcutaneously, 3 times with 1-week interval. The CT26 lysate was made by 3 times freeze-thaw cycles using liquid nitrogen. The mouse body weights were monitored at 2-day intervals. All mice were sacrificed 19 days after CT26 inoculation by cervical dislocation after exposure to saturated C02. The tumor size was measured by caliper every 2 days and the volume was calculated using the formula 0.5×(short diameter)²×(long diameter). The tumors were weighed and fixed in 10% formalin for histological analysis.

### Analysis of Ad-PD-L1ip3 Treatment-Associated Apoptosis of CT26 Cell Tumors by Immunohistochemistry

The paraffin-embedded tumor tissues were sectioned and immunostained with anti-cleaved caspase-3 antibodies. The average number of cleaved caspase-3 positive cells in 5 random fields (n=5-6) were calculated.

### Statistical Analysis

All values are expressed as the mean±standard deviation of mean. For all in vitro and in vivo experiments, statistical significance was assessed by an unpaired t-test or ANOVA followed by Tukey's test. All experiments were conducted with multiple sample determinations with several samples (n=3-6). Statistical significance was set at *, P<0.05.

### Results

### Computational Evaluation of the Interaction of PD-L1 Inhibitory Peptide and PD-L1

Using estimates of binding free energy by the MM-PBSA method, two sequences (referred to as PD-L1ip3 and PD-L1ip4, see Table 1) were selected and long explicit-solvent molecular dynamics simulations of 6 distinct docked poses of each peptide bound to human PD-L1 were performed for the validation of complex stability. We found that PD-L1ip3 remained stably bound to PD-L1 in all six cases for at least 800 ns, while PD-L1ip4 diffused away from its putative binding site within 300 ns simulation. FIG. 2A shows the conformation of the complex between PD-L1ip3 and PD-L1 with the strongest predicted binding affinity. Putative interactions between PD-L1ip3 and PD-L1 in this conformation are diagrammed in FIG. 2B. The complex appeared to be stabilized by a salt bridges between the N-terminus of the peptide and Glu58 of PD-L1, Arg3 of the peptide and Asp73, and the C-terminus of the peptide and Arg113 and Arg125. Hydrophobic contacts were noted between Lys7 and Ile8 of the peptide and Ile116 and Ala121 of PD-L1. Most of the residues of PD-L1 involved in these interactions are part of the binding interface between PD-L1 and PD-1, suggesting that this peptide might be able to compete with PD-1 for the PD-L1 binding site. This computational analysis was performed with human PD-L1 (the only experimentally derived PD-L1 structure available) at variance with the experiments, which involved murine cells; however, most of the binding site residues are identical between humans and mice, including all residues including all residues whose side chains contact with the peptide in FIG. 2B. The only exception is that Arg113 being replaced with Cys113 in mice; however, Arg125 is present in both cases so a salt bridge with the C-terminus can exist regardless. It also should be noted that the affinity of murine PD-1 for human PD-L1 is similar to that of human PD-1 for human PD-L1.

### Experimental Confirmation of Association Between the Designed Peptide and PD-L1

We experimentally verified that the computationally designed peptide, PD-L1ip3, exhibits specific binding to PD-L1. The peptide was synthesized commercially and the dissociation constant was determined by bio-layer interferometry. We found that PD-L1ip3 binds to PD-L1 with a dissociation constant of K_{D}=33±3 μM (FIG. 2C), which is only a few times weaker than the binding of PD-L1 to its natural partner PD-1 (K_{D}≈8 μM). Hence, the binding affinity of this peptide is likely sufficient to explain the biological activity of PD-L1ip3 observed in the experiments described in the following sections. The bio-layer interferometry experiments included the scrambled version of the peptide (PD-L1ip3 SC) as a negative control, which exhibited very weak binding (K_{D}≈50 mM), demonstrating that PD-L1ip3 binding to PD-L1 is not merely non-specific association.

Treatment with PD-L1ip3 Peptide or Transduction of the PD-L1ip3 Gene Did not Alter Growth of Colon Carcinoma Cells in the Absence of T Cells

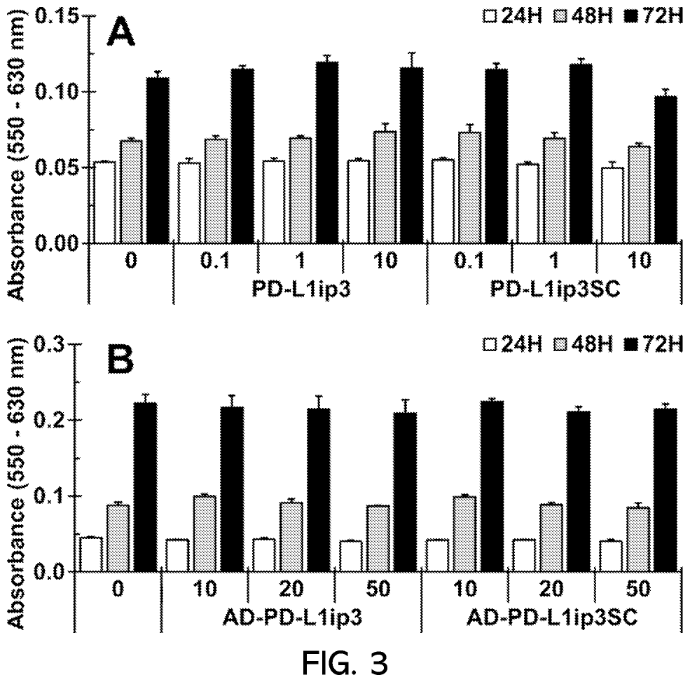

To evaluate therapeutic potential and cytotoxicity of the PD-L1ip3 peptide and transduction by Ad-PD-L1ip3, cell viability of CT26 cells was analyzed using MTT assay in the presence of mIFNγ (25 ng/ml), which was used to increase PD-L1 expression in cancer cells. The scrambled sequence version of PD-L1ip3 (PD-L1ip3SC) and the adenovirus carrying the corresponding scrambled sequence gene (Ad-PD-L1ip3SC) were used as negative controls. As shown in FIG. 3, neither treatment with the peptide form of PD-L1ip3 (0.1-10 μg/ml), nor the control peptide PD-L1ip3SC, nor gene expression by the Ad-PD-L1ip3 or Ad-PD-L1ip3SC gene (10-50 MOI) altered the cell viability of CT26 cells regardless of the pretreatment with mIFNγ. Although a slight decrease of cell viability was observed at 72 h after the treatment with 10 μg/ml PD-L1ip3SC, this decrease was not significantly different in comparison to PBS. These results indicate that both PD-L1ip3 peptide and its gene expression by adenovirus vector in cancer cells do not alter the cell viability and appears to have no inherent cytotoxicity for cancer cells in the absence of T cells.

Treatment with PD-L1ip3 Peptide or Transduction of the PD-L1ip3 Gene Significantly Increased T Cell-Induced Death of CT26 Murine Colon Carcinoma Cells

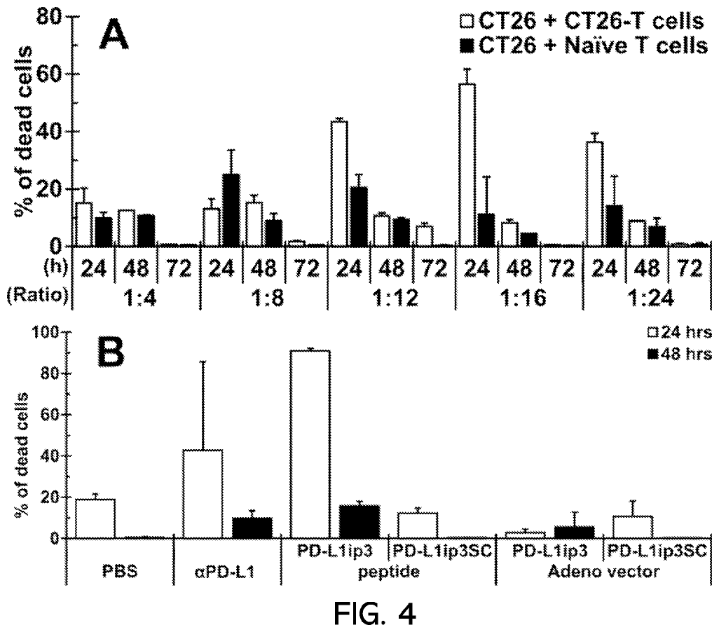

To analyze the effect of administration of PD-L1ip3 in peptide form and gene transduction by Ad-PD-L1ip3 on T cell function and cancer cell death, CT26 cell antigen-primed T cells (CT26-T cells) were generated by a treatment of mice with CT26 cell lysate and irradiated CT26 cells as described in the Methods. Preparation of the splenocytes and subsequent isolation of the CD8⁺ T cells were carried out on the same day of the co-culture study. Cell death was analyzed by a flow cytometry using live/dead staining dye. First, the most appropriate ratio of cancer cells to T cells was determined by varying their ratio from 1:4 to 1:24 (CT26 cells:T cells) in co-culture. As shown in FIG. 4A, death of CT26 cells was induced by antigen-primed T cells even within 24 h after beginning co-culture when the number of T cells was at least 12 times greater than the number of CT26 cells. The antigen-primed T cells induced death of CT26 cells to a greater extent than naïve T cells 24 h after incubation; however, this difference is less pronounced for longer times. At 48 h, the effect of antigen-primed and naïve T cells appears similar. Death of CT26 cells significantly declined 72 h after incubation in all tested ratios. On the basis of these experiments, a cancer cell to T cell ratio of 1:16 was used for all following experiments.

As shown in FIG. 4B, treatment with 10 μM of PD-L1ip3 in peptide form markedly increased the cell death of CT26 cells as compared to the PBS-treated group. However, gene transduction by Ad-PD-L1ip3 into CT26 cells and subsequent co-culture with the antigen-primed T cell increased cell death of CT26 cells only to a small extent as compared to the direct peptide treatment at 72 h after the co-culture (FIG. 4B). These results indicate that treatment with PD-L1ip3 in peptide form specifically stimulates killing of cancer cells by cancer cell antigen-primed T cells. However, the effect of gene transduction by Ad-PD-L1ip3 into CT26 cells in T cell-induced cell death in co-cultured CT26 cells is smaller than the peptide treatment and appears to require more time.

Gene Transduction by Ad-PD-L1ip3 and Combination Treatment with PD-L1ip3 Peptide Attenuated the Growth of CT26 Murine Colon Tumors in Mice

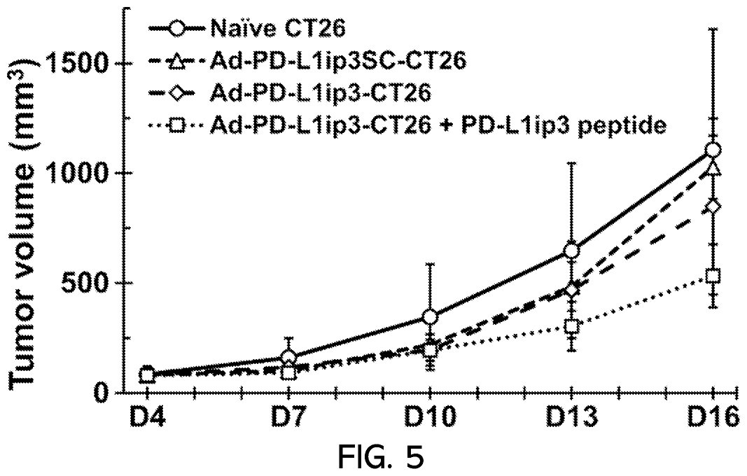

To evaluate the effect of PD-L1ip3 in vivo, we transduced the PD-L1ip3 into CT26 cells using a dosage of 100 MOI of Ad-PD-L1ip3. The cells were inoculated subcutaneously (SQ) in back of mice. In the first trial as shown in FIG. 5, Ad-PD-L1ip3 transduction into CT26 cells weakly attenuated the growth of SQ tumors (average tumor volume of 848.5±400.0 mm³, n.s.) and the effect was enhanced by combining this treatment with direct daily treatment with PD-L1ip3 in peptide form (10 mg/kg/day) (533.1±143.6 mm³, n.s.). By comparison, a larger average tumor size was measured in untreated mice (1106.3±548.8 mm³) (FIG. 5). The average tumor volume for mice receiving the scrambled sequence control vector, Ad-PD-L1ip3SC, was similar to that of untreated mice (1026.8±143.6 mm³). In this experimental scheme, the inoculation of the CT26 tumor cells initiated the host antitumor immunity, while the effect of the PD-L1 blockade by the tumor-site production of PD-L1ip3 owing to Ad-PD-L1ip3 transduction or by treatment with PD-L1ip3 in peptide form was evaluated within 3 weeks after the tumor cell inoculation. It is speculated that the host anti-tumor immunity against tumor cells was not sufficiently stimulated at this time; therefore, the PD-L1 blockade therapy in this acute allograft model appeared to be inadequate. It is suggested that a longer exposure time to tumor antigens might be more appropriate for the adequate evaluation of the PD-L1 blockade therapy by either the tumor-site production of PD-L1ip3 or in combination with daily administration of PD-L1ip3 in peptide form.

Pretreatment with CT26 Cell Lysate Enhanced Inhibition of Tumor Growth by Ad-PD-L1ip3 and the Combination Treatment with the PD-L1ip3 Peptide

We hypothesized that pretreatment with CT26 cell lysate might enhance the inhibition of CT26 tumor growth by Ad-PD-L1ip3 seen in the last section. To test this hypothesis, mice were pre-treated with CT26 cell lysate before cancer cell inoculation, 3 times separated by 1-week intervals. As shown in FIGS. 6A-6E, Ad-PD-L1ip3 transduction into CT26 cells significantly attenuated the growth of the tumors (281.4±169.6 mm³, P<0.05) and the tumor growth attenuation effect was further enhanced by the combination treatment including PD-L1ip3 in peptide form (10 mg/kg/day, every day) (172.6±60.9 mm³, P<0.05) compared to the untreated CT26 cell tumors (729.6±310.3 mm³). On the other hand, treatment with only the peptide form of PD-L1ip3 showed a minimal effect on tumor growth (607.5±220.1 mm³). These results suggest that our PD-L1 blockade treatment combining adenoviral vector-based gene therapy along with direct administration of PD-L1ip3 in peptide form is effective in inhibiting tumor growth, presumably due to the blockade of PD-L1 and PD-1 immune checkpoint mechanism.

Treatment with PD-L1ip3 in Peptide Form and Combination with Gene Transduction by Ad-PD-L1ip3 Increased Apoptotic Cells in CT26 Cell Tumors in Mice

As shown in FIG. 4, PD-L1ip3 peptide increased the cell death of CT26 cells in the presence of antigen-primed CT26-T cell in in vitro. Therefore, changes of apoptotic cell numbers in tumor nodules were evaluated by immunohistochemistry. Immunohistochemical analysis of cleaved caspase-3 positive cells in tumor nodules suggested that both monotreatment with gene transduction by Ad-PD-L1ip3 and a combination treatment with Ad-PD-L1ip3 transduction and administration of PD-L1ip3 in peptide form increased apoptotic cell numbers in tumors as compared to PBS-treated mouse tumors (FIG. 6F). However, due to the large data variation, no statistical difference between groups was observed.

### Discussion

The present study sought to design a local immune therapy by focusing on the PD-L1 blockade within tumor microenvironment using an adenovirus engineered to carry a PD-L1 inhibitory peptide gene.

To accomplish this, we designed a novel peptide for inhibiting PD-L1 using a series of computational methods, followed by experimental validation. First, we generated several candidate PD-L1 inhibitory peptide sequences using the PinaColada algorithm and the x-ray structure of the PD-L1:PD-1 complex. These sequences were then screened by creating several candidate complexes with flexible molecular docking (with the peptide near the PD-L1:PD-1 binding interface) and then estimating the binding affinity for each complex using molecular dynamics simulation (the MM-PBSA method). The two peptides with the highest estimated affinity for PD-L1 were subjected to further molecular dynamics simulations, which demonstrated the stability of complexes formed with the peptide denoted PD-L1ip3 on the sub-microsecond timescale. Experimental confirmation of the specific binding of this peptide to PD-L1 showed an affinity in the micromolar range. This unique peptide may have applications outside the scope of the present study and likely could be further optimized to obtain more potent PD-L1 inhibition.

The effect of the peptide (PD-L1ip3) on tumor growth was considered in three different ways: direct administration in peptide form, delivery of a secretory gene encoding the peptide by an adenovirus vector, and a combination of the two administration routes. These treatments were tested on a murine colon carcinoma cell line, which was evaluated in conventional cell culture and a co-culture with CT26 cell antigen-primed CD8⁺ T cells (CT26-T cells). As expected, neither direct administration of the PD-L1ip3 nor gene transduction by the adenovirus vector altered cell growth in conventional cell culture (FIG. 3). However, PD-L1ip3 in peptide form, but not gene transduction by the adenovirus vector, significantly increased CT26-T cell-dependent death of colon carcinoma cells in co-culture (FIG. 4B). This result is reasonably explained by the lower concentration of the inhibitory peptide in the culture medium due to expression of the PD-L1ip3 gene as compared to direct addition of 10 μM of the peptide. These cell culture studies, therefore, suggest that the treatment with PD-L1ip3 peptide is capable of stimulating cytotoxic T cell-induced cell death in cancer cells.

The ability of PD-L1ip3 peptide to stimulate cancer cell antigen-primed CD8⁺ T cells to induce cell death in colon carcinoma cells in co-culture studies (FIG. 4B) compelled an in vivo test of the efficacy of both PD-L1ip3 in peptide form and transduction of its gene by the Ad-PD-L1ip3 vector. In the mouse study, the effects of two mouse immune conditions, PD-L1ip3 in peptide form alone, Ad-PD-L1ip3 gene therapy alone, and a combination treatment with both direct peptide administration and gene therapy were evaluated in a subcutaneous tumor mouse model with CT26 murine colon carcinoma cells. First, the effect of direct administration of PD-L1ip3 and/or its gene expression was evaluated using syngeneic immunocompetent mice. As shown in FIG. 5, the growth of Ad-PD-L1ip3 transduced cancer cells was slower than that of un-transduced cells and this tumor growth attenuation was further pronounced by the daily direct administration of PD-L1ip3 beginning at day 7 for 13 days although tumor growth slowly continued. However, neither transduction of a scrambled sequence peptide gene by the adenovirus vector (Ad-PD-L1ip3SC) nor PD-L1ip3 in peptide form alone showed a statistically significant change in tumor growth as compared to the growth of the untreated CT26 cells. On the other hand, when mice were previously treated with CT26 cell lysate three times for two weeks, mimicking chronic tumor antigen exposure, growth of Ad-PD-L1ip3 transduced cancer cells was significantly slower than that of un-transduced cells and this tumor growth was almost completely inhibited by the co-treatment with daily administration of PD-L1ip3 in peptide form starting at day 7 for 13 days. As expected, a sole treatment consisting of transduction by Ad-PD-L1ip3SC, a vector encoding the scrambled sequence control peptide, showed no significant change in tumor growth as compared to the untreated group. The tumor growth pattern for individual mice in each group (FIGS. 6B-6E) indicates clear efficacy of co-treatment with the Ad-PD-L1ip3 vector and PD-L1ip3 in peptide form. These results clearly indicate a practical possibility of tumor cell-targeted gene therapy with a PD-L1 inhibitory peptide gene along with direct peptide administration. Since the peptide showed no cytotoxicity (FIG. 3) and the activation of CD8⁺ T cells occurs in cell culture, this method is considered to be a safe immune checkpoint therapy. However, fate of the PD-L1ip3-activated T cells and overall safety await further study.

The discrepancy between the relatively small effect of PD-L1ip3 gene expression on cancer cell death in the co-culture study (FIG. 4B) versus its considerable effect in the mouse study (FIGS. 5 and 6) may be explained by the difference in the participating cell types and the duration of the study. The cell culture study included only two cell types, effector T cells and cancer cells, whereas other cell types including natural killer cells (NK cells) are potentially participating in cancer cell death during the mouse study. Second, the T cell-induced death of cancer cells was monitored only 72 h after gene transduction by Ad-PD-L1ip3 in the co-culture study, whereas the tumor growth effect was monitored for two weeks in the mouse study. In support of the involvement of the PD-L1/PD-1 immune checkpoint mechanism in other immune cells, a recent report describes the importance of PD-1⁻ NK cells in anti-cancer immunity for mouse cancer models and human lung cancer. Moreover, blockade of PD-L1⁺ NK cells by an anti-PD-L1 antibody is shown to increase therapeutic efficacy in human patients, suggesting that PD-L1 in NK cells is an inhibitory molecule in NK cell-induced oncolysis. Therefore, determination of potential targets for this PD-L1 inhibitory peptide must be studied thoroughly.

### Conclusion

The computationally designed peptide, PD-L1ip3, was demonstrated to bind to PD-L1 in experiment. Treatment with this peptide alone showed negligible cytotoxicity, but stimulated cancer antigen-primed CD8⁺ T cells in an oncolysis assay. Transduction of the PD-L1ip3 gene by an adenovirus vector into CT26 colon carcinoma cells significantly attenuated tumor growth in mice when their immune systems were previously stimulated by CT26 cell lysate. Tumor growth of PD-L1ip3-expressing CT26 cells was almost completely inhibited by a combination treatment of direct administration of PD-L1ip3 in peptide form along with an adenovirus gene therapy encoding the same peptide. Although further studies are required to confirm potential target immune cells and the safety of PD-L1ip3 by orthodox pharmacokinetics, pharmacodynamics, and multispecies toxicity studies, this data shows that this peptide therapy could be a locally effective agent that can stimulate antitumor immunity, thereby inhibiting colon cancer growth.

### Abbreviations

PD-1, Programmed death-1; PD-L1, Programmed death-ligand-1; PD-L1ip3, PD-L1 inhibitory peptide 3; PD-L1ip3SC, Scrambled analogue of PD-L1ip3; CRC, Colorectal cancer; MSI, Microsatellite instability; dMMR, DNA mismatch repair gene defects, RPMI, Roswell Park Memorial Institute; FBS, Fetal bovine serum; MEM, Eagle's minimum essential medium; PBS, Phosphate-buffered saline; MM-PBSA, Molecular mechanics-Poisson-Boltzmann and surface area; NAMD, Nanoscale molecular dynamics; Ad-PD-L1ip3, PD-L1ip3 gene encoding adenovirus vector; MD, Molecular dynamics; RT-qPCR, Reverse transcription quantitative polymerase chain reaction; MOI, Multiplicity of infection; MTT, 3-(4,5-dimethylthiazol-2-yl)-2,5-diphenyltetrazolium bromide; SQ, Subcutaneous; LPS, Lipopolysaccharides; IV, Intravascular; GFP, Green fluorescent protein; IFNγ, Interferon gamma; ANOVA, Analysis of variance; KD, Kilodalton; CT26-T cell, CT26 cell antigen-primed T cells; CTLA-4, Cytotoxic T-lymphocyte-associated protein 4; NK cells, Natural killer cells

### Example 2

### Lung Cancer

PD-L1-specific inhibitory peptide blocks the PD-L1 and PD-1 interaction, thus disrupting the downregulation of CTL response to lung cancer cells and activating anti-cancer cell immunity with a high degree of specificity. This hypothesis will be tested by determining cell death of lung cancer cells in the lung cancer and T lymphocyte co-culture system in the presence or absence of the inhibitory peptide.

### Materials and Methods

### Cell Lines

H1299 human non-small cell lung carcinoma cell line (ATCC)
Jurkat cells (Human T lymphoblasts, ATCC)
Primary cultured murine CD8⁺ T cell primed with H1299 cell lysate (H1299 cell antigens)

### Analysis

Flow cytometry analysis for the cytotoxicity of antigen-specific T cell toward cancer cell populations
   Co-Culture of Activated Jurkat Cells with H1299 Lung Cancer Cells in the Presence of PD-L1 Inhibitory Peptide Slightly Increased Cell Death in H1299 Cells.

Co-culture assay procedure. Dead cell populations were measured by flow cytometry at 48 hours post-co-culture using propidium iodide (PI) staining. After 48 hour co-culture, fluorescent microscopy showed physical association of Jurkat cells and GFP⁺ H1299 cells, suggesting an interaction between these cells (not shown). Flow cytometric analysis showed only minor increases of cell death in H1299 cells treated with PD-L1ip3 scrambled and PD-L1ip3 at 48 hours after co-culture of two cell types (not shown).

### Antigen-Specific T Cell Generation and Co-Culture Assay

Co-Culture of H1299 Cell Antigen-Specific T Cells with H1299 Lung Cancer Cells in the Presence of PD-L1 Inhibitory Peptide Increased Cell Death in H1299 Cells.

Mice were immunized with H1299 cells via tail vein injection. T cells were extracted from spleen, purified, and expanded by co-culturing with dendritic cells (DCs) stimulated with H1299 cell lysate. Expanded T cells were used in co-culture assay. Flow cytometric analysis showed clear increases of cell death in H1299 cells treated with PD-L1 inhibitory peptide more than scrambled peptide at 48 hours after co-culture of two cell types.

### Discussion

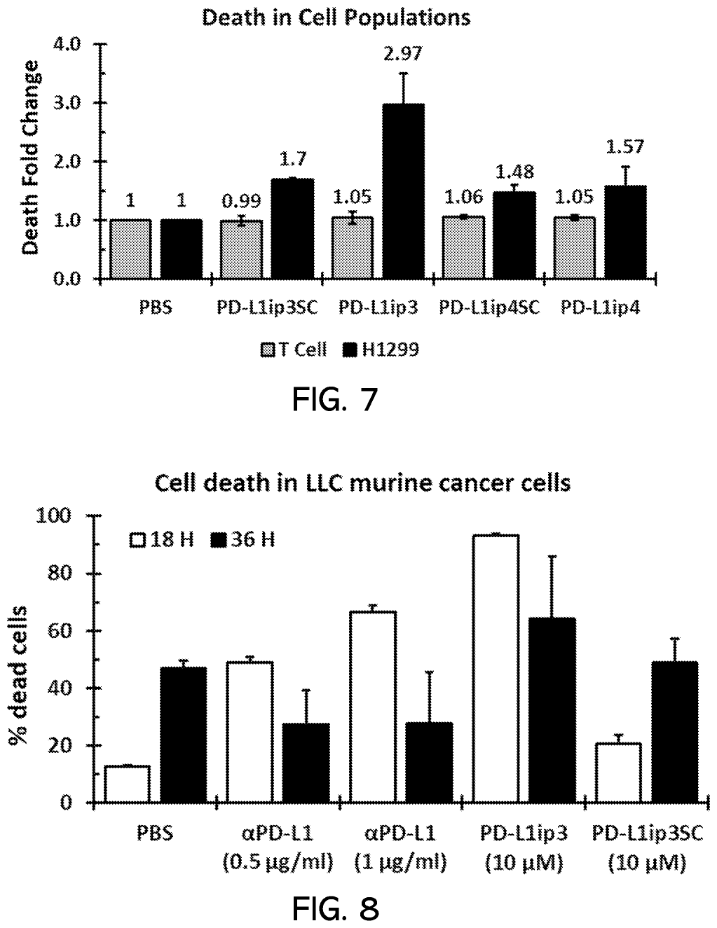

Preliminary data indicates that PD-L1ip3 and scrambled peptide treatments caused a 30% increase in cell death in H1299 cancer cells when co-cultured with nonspecifically activated Jurkat cells (not shown). However, PD-L1ip but not scrambled peptide treatment caused a 50% increase in cell death in H1299 cancer cells when co-cultured with H1299 cell antigen-primed T lymphocytes (FIG. 7). PD-L1ip4 peptide did not alter T cell-dependent death in both assays. The current study suggests that inhibitory peptide PD-L1ip3 could be a useful to block PD-1 and PD-L1 binding, thereby inhibiting PD-1 immune checkpoint and increasing T cell-dependent cancer cell death.

### Example 3

### PD-L1ip3 Increased the Cytotoxicity of Cancer Cell Antigen Primed CD8⁺ T Cells for LLC Murine Lung Cancer

In this cell culture study, the cytotoxicity of antigen-specific CD8⁺ T cells toward murine LLC lung carcinoma cells was enhanced, which was as good as or better than the positive control (anti-PD-L1 antibody). See FIG. 8.

### Methods

Generation of LLC cell antigen primed CD8⁺ T cells in vivo: Mice were subcutaneously injected with LLC lysate (0.5×10⁶ cells/mouse in 100 μl) at Day 0. X-ray (100 Gy) irradiated LLC cells were cocultured with JAWSII immature dendritic cells (JAWS-irrLLC) at a 1:1 ratio for 48 hrs in the presence of LPS (1 μg/ml). The cells were then intravenously injected into mice via tail vein (0.5×10⁴ cells/mouse in 200 μl) at Day 7. At Day 21, the splenocytes were harvested and CD8⁺ T cells (AP-CD8⁺ T cells) were isolated using commercially available isolation kit.

Evaluation of T cell-induced death of lung carcinoma cells in co-culture: Permanently GFP-expressing LLC cells (1×10⁴ cells) were treated with murine IFNγ (25 ng/ml) for 48 hrs. The cells were treated with 10 μM PD-L1ip3. PD-L1ip3SC (10 μM) and mouse anti-PD-L1 antibody (αPD-L1: 0.5 or 1.0 μg/ml) were used as control. The cells were then cocultured with antigen primed CD8⁺ T cells 1:16 ratio. The cytotoxicity of AP-CD8⁺ T cells toward LLC cells at 18 and 36 hrs after coculture was determined by flow cytometry.

### Example 4

### Effect of AD-PD-L1ip on Survival of CT26 Tumor Bearing Mouse

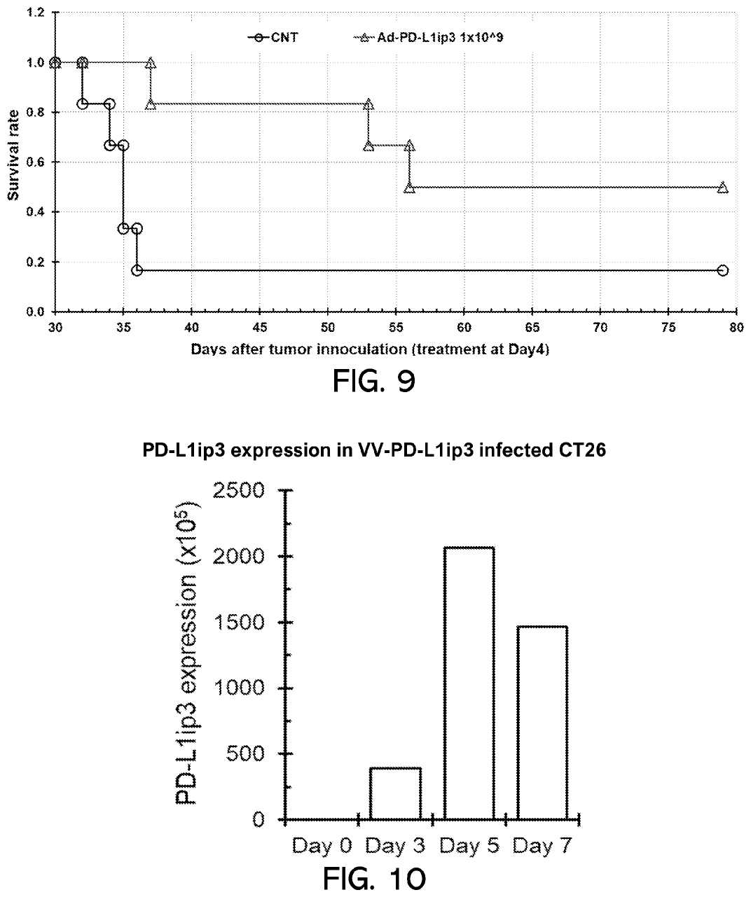

This study demonstrates survival of the mice beyond 40 days (even out to 80 days), when treated with adenovirus vector encoding the inhibitory peptide sequence. See FIG. 9.

Mice were pretreated with CT26 lysate (1×10⁶ cells/100 μl, SQ, once a week) 3 times. One week after last lysate injection, 2.5×10⁵ CT26 cells were intraperitoneally (IP) injected into mice. The mice were treated with AD-PD-L1ip3 or AD-PD-L1ip3SC (1×10⁹ PFU, IP) 4 days after cancer cell injection.

### Example 5

### Vaccinia Virus Vector Encoding PD-L1ip3 Sequence

### Generation of Replication-Limited Mutant Vaccinia Virus (VV) Vectors Secreting PD-L1 Inhibitory Peptide (PD-L1ip)

A cancer-targeted recombinant VV encoding the PD-L1ip3 gene was generated as described. Briefly, VV Western Reserve (WR) strain genome lacking thymidine kinase (TK) and viral growth factor (VGF) (ddVV) was used. Knockout of TK and VGF genes attenuates the virus's virulence and improves its cancer cell target specificity. The TK and VGF knockout ddVV can replicate and lyse its infected cancer cells, which provides another mechanism to hinder the cancer cell growth in addition to the production of PD-L1ip3.

In more detail, first, the viral thymidine kinase (TK) and viral growth factor (VGF) genes on VV genome were deleted through homologous recombination. It has been shown that a VV WR strain lacking the TK and VGF genes prefers to replicate in cancer cells, but not in normal animal cells in the resting state. Then the DNA sequence coding for the N-terminal secretion signal from the V-J2-C region of the mouse Ig κ-chain (METDTLLLWVLLLWVPGSTGD, SEQ ID NO:15), a short linker (AAQPARRA, SEQ ID NO: 16), a Myc tag (SEQ ID NO:17), and PD-L1ip3 (GTRLKPLIICVQWPGL, SEQ ID NO:1) was amplified by PCR and inserted at the TK locus through homologous recombination. As a negative control, the above procedure was repeated with a scrambled PD-L1ip3 (PD-L1ip3 SC) sequence. Correct DNA insertion was verified by PCR and DNA sequencing. Although these vectors produce longer peptides than the originally designed PD-L1ip3 or PD-L1ip3SC due to the linker and Myc tag, additional simulations revealed that even greater affinity for the complete linker+Myc+PD-L1ip peptide. Although these vectors produce longer peptides than the originally designed PD-L1ip3 or PD-L1ip3SC due to the linker and Myc tag, additional simulations revealed that even greater affinity for the complete linker+Myc+PD-L1ip peptide.

### Evaluation of PD-L1ip3 Expression in VV-PD-L1ip3 Infected CT26 Cells

CT26 cells (1×10⁵ cells/well) were seeded into 12-well plate. At 24 hrs after cell seeding, the cells were infected with VV-PD-L1ip3 with 0.1 multiplicity of infection (MOI). Total RNA was extracted at day 3, 5 and 7 after infection and PD-L1ip3 expression was measured by real-time PCR. As shown in FIG. 10, gene expression by VV vector is approximately 50 times higher in the peak expression and 500 time higher expression on day 7 as compared to those by adenovirus vector. Gene expression by VV vector lasted much longer than that by the adenovirus vector.

### Evaluation of VV-PD-L1ip3 Treatment on the Growth of CT26 Tumor in Mice

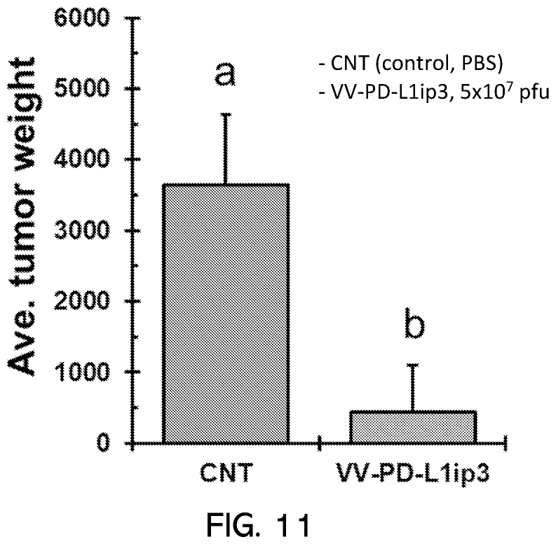

5×10⁵ CT26 murine colon carcinoma cells were inoculated intraperitoneally (IP) (200 μl in PBS) into mice. The mice were treated with VV-PD-L1ip3 (5×10⁷ PFU, IP) 4 days after cancer cell injection. Mice were euthanized 35 days after cancer cell injection. The results are shown in FIG. 11.

1. An immune checkpoint inhibitor oligopeptide comprising the sequence:
PD-L1ip3:
(SEQ ID NO: 1)
GTRLKPLIICVQWPGL.

2. A nucleic acid encoding an immune checkpoint inhibitor oligopeptide according to claim 1.

3. A recombinant viral vector comprising a nucleic acid according to claim 2.

4. A cell comprising an immune checkpoint inhibitor oligopeptide according to claim 1, or nucleic acid encoding said oligopeptide, or viral vector comprising said nucleic acid.

5. A therapeutic composition comprising a therapeutically effective amount of an immune checkpoint inhibitor oligopeptide according to claim 1, a nucleic acid encoding said oligopeptide, a viral vector comprising said nucleic acid, or a cell comprising said oligopeptide, nucleic acid, or viral vector, dispersed in a pharmaceutically-acceptable carrier.

6. A method of treating cancer, said method comprising administering a therapeutically effective amount of an immune checkpoint inhibitor oligopeptide according to claim 1, a nucleic acid encoding said oligopeptide, a viral vector comprising said nucleic acid, or a cell comprising said oligopeptide, nucleic acid, or viral vector to a subject in need thereof.

7. The method of claim 6, wherein a composition comprising a therapeutically effective amount of said immune checkpoint inhibitor oligopeptide, nucleic acid, viral vector, or cell is administered to said subject.

8. The method of claim 6, wherein a therapeutically effective amount of said immune checkpoint inhibitor oligopeptide is administered to said subject.

9. The method of claim 8, further comprising co-administering a therapeutically effective amount of said nucleic acid, viral vector, or cell to said subject, wherein said immune checkpoint inhibitor oligopeptide is expressed by said nucleic acid, viral vector, or cell in said subject after said administration.

10. The method of claim 6, wherein said immune checkpoint inhibitor oligopeptide, nucleic acid, viral vector, or cell is locally administered in or near a cancerous site of said subject.

11. The method of claim 6, wherein said immune checkpoint inhibitor oligopeptide, nucleic acid, viral vector, or cell enhance an anti-tumor immune response against cancer cells in said subject.

12. The method of claim 6, wherein said immune checkpoint inhibitor oligopeptide, nucleic acid, viral vector, or cell stimulate an anti-tumor immune response against cancer cells in said subject.

13. The method of claim 6, wherein said immune checkpoint inhibitor oligopeptide, nucleic acid, viral vector, or cell are administered intramuscularly, intraperitoneally, subcutaneously, intradermally, intravenously, mucosally, topically, intratracheally or intratumorally.

14. The method of claim 6, wherein said immune checkpoint inhibitor oligopeptide, nucleic acid, viral vector, or cell are coadministered with other immunotherapies and/or chemotherapies.

15. The method of claim 6, wherein said immune checkpoint inhibitor oligopeptide, nucleic acid, viral vector, or cell are administered as a primary treatment.

16. The method of claim 6, wherein said immune checkpoint inhibitor oligopeptide, nucleic acid, viral vector, or cell are administered as an adjunctive treatment to a primary treatment comprising chemotherapy, radiation therapy, hormone therapy, and/or immunotherapy.

17. The method of claim 6, wherein said immune checkpoint inhibitor oligopeptide, nucleic acid, viral vector, or cell facilitate an increased immune response in said subject for at least 24 hours after administration.

18. The method of claim 6, wherein said subject exhibits a reduction in cancer cell numbers, a reduction in tumor nodule numbers, a reduction in volume of cancer cells, a reduction in tumor volume, and/or a reduction in rate of growth of said cancer cells or tumor after said administration.

19. A kit comprising an immune checkpoint inhibitor oligopeptide according to claim 1, a nucleic acid encoding said oligopeptide, a viral vector comprising said nucleic acid, or a cell comprising said oligopeptide, nucleic acid, or viral vector in a unit dosage form in a container, and instructions for administering said immune checkpoint inhibitor oligopeptide, nucleic acid, viral vector, or cell to a subject in need thereof.

20. (canceled)

21. A medicament for use in treating cancer, inhibiting growth of cancer cells, or enhancing cancer treatment, comprising an immune checkpoint inhibitor oligopeptide according to claim 1, a nucleic acid encoding said oligopeptide, a viral vector comprising said nucleic acid, or a cell comprising said oligopeptide, nucleic acid, or viral vector.
